(19) United States
(12) Patent Application Publication
(10) Pub. No.: US 2004/0220165 A1
(43) Pub. Date: Nov. 4, 2004

(54) Process for asymmetrically hydrogenating keto carboxylic esters

(76) Inventors: John Meurig Thomas, Cambridge (GB); Brian F. G. Johnson, Cambridge (GB); Robert Raja, Cambridge (GB); Matthew David Jones, Cambridge (GB)

Correspondence Address: LANXESS CORPORATION, PATENT DEPARTMENT/ BLDG 14, 100 BAYER ROAD, PITTSBURGH, PA, 15205-9741, US

(21) Appl. No.: 10/772,198

(22) Filed: Feb. 4, 2004

(30) Foreign Application Priority Data
Feb. 12, 2003 (DE) 10305943.1
Jun. 16, 2003 (DE) 10326915.0

(51) Int. Cl. A61K 31/555; A61K 9/14

(52) U.S. Cl. 514/184; 424/489

(57) ABSTRACT

The present invention relates to a process for preparing enantiomerically enriched α- and β-hydroxycarboxylic esters from the corresponding ketocarboxylic esters and also to catalysts usable therefor.

### BACKGROUND OF THE INVENTION

1. Field of the Invention

The present invention relates to a process for preparing enantiomerically enriched α- and β-hydroxy carboxylic esters from the corresponding keto carboxylic esters and also relates to catalysts usable therefor.

2. Brief Description of the Prior Art

Enantiomerically enriched α- and β-hydroxy carboxylic esters are valuable reagents for optical resolution and important intermediates in the preparation of pharmaceuticals and agrochemicals. Customarily, enantiomerically enriched α- and β-hydroxy carboxylic esters are obtained by the process of catalytically hydrogenating the corresponding α- and β-keto carboxylic esters, usually using transition metal complexes having chiral phosphines as ligands as catalysts (see, for example, Genet et al., Tetrahedron, Asymmetry, 1994, 5(4), 675-690).

A disadvantage of chiral phosphines is their high cost and oxidation sensitivity, which is why they are used on the industrial scale predominantly in homogeneous processes, if at all.

Alternatively, processes using platinum or nickel catalysts modified by cinchona alkaloids or tartaric acid derivatives are known (T. Mallat et al., Fine Chemicals through Heterogeneous Catalysis, Wiley-VCH, 2001, p. 449 ff).

Also, Ferrand et al. (Tetrahedron: Asymmetry, 13, 2002, pp. 1379 to 1384) describe the use of rhodium, ruthenium and iridium complexes with chiral diamines for the hydrogenation of keto esters.

A common disadvantage to all these processes is that they provide at best a moderate enantiomeric excess.

There was, therefore, a need to provide catalysts which make possible high yields and enantioselectivities, in particular in a process for preparing enantiomerically enriched α- and β-hydroxy carboxylic esters.

### SUMMARY OF THE INVENTION

In accordance with the foregoing, the present invention encompasses substances which comprise at least

one micro-, meso- or macroporous support material and

compounds, adsorbed thereon or therein, of the formula (I)

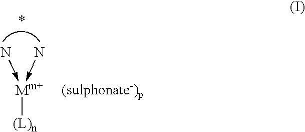

where

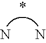

is an enantiomerically enriched chiral nitrogen compound,

(M^{m+}) is a metal having valency m

L is an anionic or uncharged ligand

(sulphonate⁻) is the anion of a sulphonic acid and

p is one or two and

n is one, two, three or four,

with the proviso that m−p−[number of anionic ligands]=0.

For the purposes of the invention, enantiomerically enriched compounds are enantiomerically pure compounds or mixtures of enantiomers of a compound in which one enantiomer is present in an enantiomeric excess, (also referred to hereinbelow as ee, relative to the other enantiomer). Preferably, this enantiomeric excess is 10 to 100% ee, particularly preferably 90 to 100% ee and very particularly preferably 95 to 100% ee.

For the purposes of the invention, all radical definitions, parameters and illustrations hereinabove and listed hereinbelow, in general or within areas of preference, i.e. the particular areas and areas of preference, may be combined as desired.

### DETAILED DESCRIPTION OF THE INVENTION

Alkyl, alkoxy, alkylene and alkenylene hereinbelow are each independently a straight-chain, cyclic, branched or unbranched alkyl, alkoxy, alkylene and alkenylene radical respectively, each of which may optionally be further substituted by C₁-C₄-alkoxy. The same applies to the nonaromatic moiety of an arylalkyl radical.

Illustrative but non-limiting examples of the radicals are as follows. C₁-C₄-Alkyl is, for example, methyl, ethyl, n-propyl, isopropyl, n-butyl, sec-butyl and tert-butyl, C₁-C₈-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 1-ethyl-2-methylpropyl, n-heptyl and n-octyl, and C₁-C₂₀-alkyl is further additionally, for example, adamantyl, the isomeric menthyls, n-nonyl, n-decyl and n-dodecyl.

C₁-C₄-Alkoxy is, for example, methoxy, ethoxy, n-propoxy, isopropoxy, n-butoxy, sec-butoxy and tert-butoxy, C₁-C₈-alkoxy is additionally, for example, n-pentoxy, 1-methylbutoxy, 2-methylbutoxy, 3-methylbutoxy, neopentoxy, 1-ethylpropoxy, cyclohexoxy, cyclopentoxy, n-hexoxy and n-octoxy, and C₁-C₂₀-alkoxy is further additionally, for example, adamantoxy, the isomeric menthoxy radicals, n-decoxy and n-dodecoxy.

C₁-C₄-Alkylene is, for example, methylene, 1,1-ethylene, 1,2-ethylene, 1,1-propylene, 0,1,3-propylene, 1,4-butylene, and C₁-C₈-alkylene is additionally, for example, 1,2-cyclohexylene and 1,2-cyclopentylene.

C₂-C₈-Alkenylene is, for example, 1,1-ethenylene 2-ethoxy-1,1-ethenylene and 2-methoxy-1,1-ethenylene.

Haloalkyl, haloalkoxy and haloalkylene are each independently a straight-chain, cyclic, branched or unbranched alkyl radical and alkylene radical respectively, each of which is singly, multiply or fully substituted by halogen atoms.

For example, C₁-C₂₀-haloalkyl is trifluoromethyl, chloromethyl, 2-chloroethyl, 2,2,2-trifluoroethyl, pentafluoroethyl, nonafluorobutyl, heptafluoroisopropyl, perfluorooctyl, perfluorodecyl and perfluorohexadecyl.

Aryl is in each case independently a heteroaromatic radical having 5 to 14 framework carbon atoms of which no, one, two or three framework carbon atoms per cycle, but at least one framework carbon atom in the entire molecule, may be substituted by heteroatoms selected from the group of nitrogen, sulphur or oxygen, or and is preferably a carbocyclic aromatic radical having 6 to 14 framework carbon atoms.

Examples of carbocyclic aromatic radicals having 6 to 14 framework carbon atoms are, for example, phenyl, biphenyl, naphthyl, phenanthrenyl, anthracenyl or fluorenyl, heteroaromatic radicals having 5 to 14 framework carbon atoms of which no, one, two or three framework carbon atoms per cycle, but at least one framework carbon atom in the entire molecule, may be substituted by heteroatoms selected from the group of nitrogen, sulphur or oxygen are, for example, pyridinyl, oxazolyl, benzofuranyl, dibenzofuranyl or quinolinyl.

The carbocylic aromatic radical or heteroaromatic radical may also be substituted by up to five identical or different substituents per cycle which are selected, for example, from the group of nitro, cyano, chlorine, fluorine, C₁-C₁₂-alkyl, C₁-C₁₂-haloalkyl, C₁-C₁₂-haloalkoxy, C₁-C₁₂-haloalkylthio, C₁-C₁₂-alkoxy, di(C₁-C₈-alkyl)amino or tri(C₁-C₆-alkyl)siloxyl be substituted.

Arylene is an aryl radical which has a further bonding site on the aromatic framework and is therefore divalent.

Arylalkyl is in each case independently a straight-chain, cyclic, branched or unbranched alkyl radical as defined above which may be singly, multiply or fully substituted by aryl radicals as defined above.

Arylalkylene is an arylalkyl radical which has a further bonding site on the aromatic framework and is therefore divalent.

Areas of preference for the substances according to the invention are defined hereinbelow:

Preferred support materials have a pore size in the range from 15 to 250 Å, more preferably in the range from 20 to 100 Å. The terms micro-, meso- and macroporous, and the nomenclature of the zeolites as used herein are to be interpreted in accordance with IUPAC (McCusker et al. Pure Appl. Chem, vol. 73, No. 2, pp. 381-394, 2001). Examples of suitable support materials include silica gels, or zeolites of the Davison, MOR, X, Y, MCM, ZSM, FAU, MFI, L, BEA, FER, A and SBA type or those of the AIPO, MAIPO and SAPO type, and the zeolites mentioned may optionally be isomorphically substituted. Particular preference is given to support materials of the MCM or Davison type, for example MCM-41 (approx. 30 Å), Davison 923 (approx. 22 Å), Davison 634 (approx. 60 Å).

In formula (I)

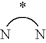

is preferably enantiomerically enriched chiral nitrogen compounds of the formula (II)

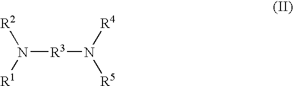

where

R¹, R², R⁴ and R⁵ are each independently hydrogen, C₁-C₈-alkyl, C₅-C₁₅-arylalkyl or C₄-C₁₄-aryl, or NR¹R² and/or NR⁴R⁵ which as a whole is a cyclic amino radical having a total of 4 to 20 carbon atoms,

R³ is a divalent radical having a total of 2 to 30 carbon atoms or

R³ and at least one of the radicals R¹, R², R, R⁵ together are part of a cyclic amino radical having a total of 4 to 20 carbon atoms.

Preferred compounds of the formula (II) are those in which

R¹, R², R⁴ and R⁵ are each independently hydrogen, C₁-C₈-alkyl, C₅-C₁₅-arylalkyl or C₄-C₁₄-aryl, or NR¹R² and/or NR⁴R⁵ which as a whole is a 5- or 6-membered monocyclic amino radical which is optionally mono-, di-, tri- or tetrasubstituted on the carbon framework by C₁-C₄-alkyl and

R³ is a divalent radical which is selected from the group of C₂-C₈-alkylene which may optionally be further mono- or disubstituted by C₄-C₁₄-aryl radicals, C₅-C₁₅-arylalkylene, C₄-C₁₄-arylene or bis(C₄-C₁₄-arylene) or

R³ and one of the radicals R¹, R², R⁴ and R⁵ together are part of a 5- or 6-membered monocyclic amino radical which is optionally additionally mono-, di-, tri- or tetrasubstituted on the carbon framework by C₁-C₄-alkyl.

Particularly preferred compounds of the formula (II) are those in which

R¹, R², R⁴ and R⁵ are each independently hydrogen, methyl or ethyl and

R³ is a divalent radical which is selected from the group of 1,2-bis(C₄-C₁₄-aryl)-1,2-ethylene, 1,2-cyclohexylene, 1,1′-2,2′-bis(C₄-C₁₄-arylene) or

R³ and one of the radicals R¹, R², R⁴ and R⁵ together are part of a pyrrolidinyl or piperidinyl radical.

Very particularly preferred compounds of the formula (II) are (1R,2R)-1,2-diphenylethylenediamine, (1S,2S)-1,2-diphenylethylenediamine, (1R,2R)-1,2-dimethylethylenediamine, (1S,2S)-1,2-dimethylethylenediamine, (1R,2R)-1,2-cyclohexylenediamine, (1S,2S)-1,2-cyclohexylenediamine, (S)-2-aminomethyl-1-ethylpyrrolidine, (R)-2-aminomethyl-1-ethylpyrrolidine, (S)-(2-pyrrolidinylmethyl)pyrrolidine, (R)-(2-pyrrolidinylmethyl)pyrrolidine, (S)-2-aminomethyl-1-methylpyrrolidine, (R)-2-aminomethyl-1-methylpyrrolidine, (R)-1,1′-diamino-2,2′-binaphthyl, (S)-1,1′-diamino-2,2′-binaphthyl, (R)-1,1 diamino-6,6′-dimethoxy-2,2′-biphenyl and (S)-1,1′-diamino-6,6′-dimethoxy-2,2′-biphenyl, and even greater preference is given to (R)-2-aminomethyl-1-ethylpyrrolidine, (S)-(2-pyrrolidinylmethyl)pyrrolidine, (R)-(2-pyrrolidinylmethyl)pyrrolidine and (S)-2-aminomethyl-1-methylpyrrolidine.

Also in formula (I),

(M^{m+}) is preferably cobalt in the formal oxidation states 0, +2 and +3, rhodium and iridium in the formal oxidation states +1 and +3, nickel, palladium and platinum in the formal oxidation states 0 and +2 and also ruthenium in the formal oxidation state +2, and preference is given to Rh^{I}, Ir^{I} and Pd^{II}.

L is preferably the following ligand types: monoolefins, for example ethylene, cyclooctene and cyclohexene, diolefins, for example 1,5-cyclooctadiene (cod), norbornadiene (nbd) and butadiene, nitriles such as acetonitrile (ACN), benzonitrile and benzylnitrile, aromatics such as benzene, mesitylene and cymene, and also anionic ligands such as allyl, methylallyl, phenylallyl, C₁-C₈-alkyl acylacetonates, C₁-C₈-alkyl acylates, chloride, bromide and iodide.

(sulphonate⁻) is preferably salts of the type R₆SO₃— where R⁶ is C₁-C₁₂-alkyl, C₁-C₂₀-haloalkyl, C₄-C₁₄-aryl or C₅-C₁₅-arylalkyl. R⁶ is preferably methyl, phenyl, p-tolyl and C₁-C₂₀-perfluoroalkyl, particularly preferably C₁-C₄-perfluoroalkyl, in particular trifluoromethyl.

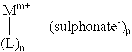

as an entire fragment is particularly preferably Rh(cod)OTf, Ir(cod)OTf, Rh(nbd)OTf, Ir(nbd)OTf, Pd(allyl)OTf, Rh(cod)OMes, Ir(cod)OMes, Rh(nbd)OMes, Ir(nbd)OMes, Pd(allyl)OMes, Rh(cod)ONf, Ir(cod)ONf, Rh(nbd)ONf, Ir(nbd)ONf and Pd(allyl)ONf, where OTf is trifluoromethanesulphonate, OMes is methanesulphonate and ONf is nonafluorobutanesulphonate.

Very particularly preferred compounds of the formula (I) are those of the formulae (Ia), (Ib), (Ic), (Id), (le) and (If)

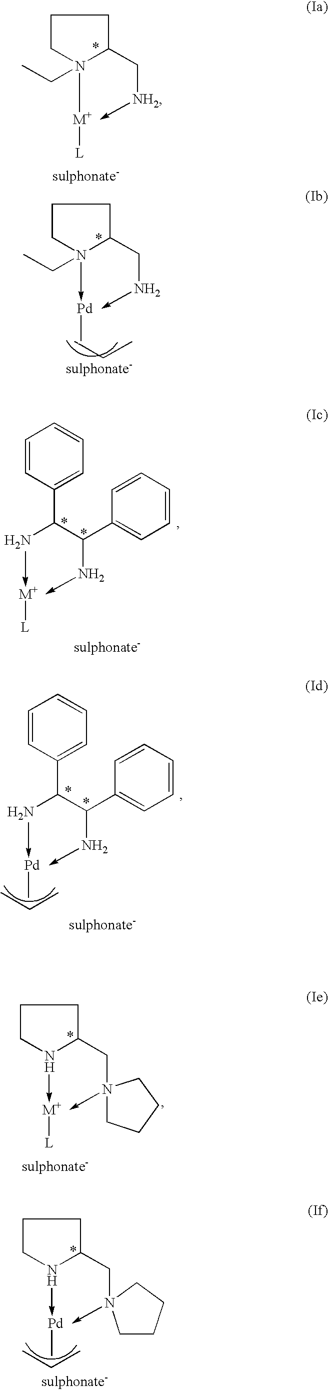

where, in each case,

* marks a stereogenic centre which is either R- or S-configured, with the proviso that mesoforms are excluded (compounds of the formula (Ic) and (Id))

M⁺ is rhodium^{I} or iridium^{I} and

L is cod or nbd and

sulphonate⁻ is trifluoromethanesulphonate, mesylate or nonafluorobutanesulphonate.

The compounds of the formula (I) are likewise encompassed by the invention, with the exception of the following:

[Rh(cod)((S)-2-aminomethyl-1-ethylpyrrolidine)]OTf and [Rh(cod)((1R,2R)-1,2-diphenylethylenediamine)] OTf.

The compounds of the formula (I), in particular those of the formulae (Ia) to (If), can be prepared in a manner known per se, for example, by reacting enantiomerically enriched chiral nitrogen compounds of the formula (II) with transition metal compounds, preferably in the presence of an organic solvent.

Useful organic solvents for the reaction are typically aliphatic or aromatic, optionally halogenated hydrocarbons, for example petroleum ether, benzene, toluene, the isomeric xylenes, chlorobenzene, the isomeric dichlorobenzenes, hexane, cyclohexane, dichloromethane or chloroform, and also preferably ethers, such as diethyl ether, diisopropyl ether, dioxane, tetrahydrofuran, methyl tert-butyl ether or ethylene glycol dimethyl ether or ethylene glycol diethyl ether. Particularly preferred organic solvents are toluene, diethyl ether, tetrahydrofuran and methyl tert-butyl ether.

Preferred transition metal compounds for the reaction with enantiomerically enriched chiral nitrogen compounds of formula (II) are those of the formula (IIIa)

M¹(An¹)ₚ₁ (IIIa)

where

M¹ is ruthenium, rhodium, iridium, nickel, palladium or platinum and

An¹ is halide and

P1 for ruthenium, rhodium and iridium is 3, and

for nickel, palladium and platinum is 2,

or transition metal compounds of the formula (IIIb)

M²(An²)ₚ₂L¹₂ (IIIb)

where

M² is ruthenium, rhodium, iridium, nickel, palladium or platinum and

An² is halide or a sulphonate,

p2 for rhodium and iridium is 1, and

for nickel, palladium, platinum and ruthenium is 2 and

L¹ is in each case a C₂-C₁₂-alkene, for example ethylene or cyclooctene, or a nitrile, for example acetonitrile, benzonitrile or benzyl nitrile, or

L¹₂ together is a (C₄-C₁₂)-diene, for example norbornadiene or 1,5-cyclooctadiene,

or transition metal compounds of the formula (IIIc)

[M³L²An³₂]₂ (IIIc)

where

M³ is ruthenium and

L² is cod, nbd, allyl, methylallyl or aryl radicals, for example cymene, mesitylene, benzene and

An³ is halide or sulphonate,

or transition metal compounds of the formula (IIId)

M⁴ₚ₃[M⁵(An³)₄] (IIId),

where

M⁵ is palladium, nickel, iridium or rhodium and

An³ is chloride or bromide and

M⁴ is lithium, sodium, potassium, ammonium or organic ammonium and

P3 for rhodium and iridium is 3, and

for nickel, palladium and platinum is 2,

or transition metal compounds of the formula (IIIe)

[M⁶(L³)₂]An⁴ (IIIe)

where

M⁶ is iridium or rhodium and

L³ is a (C₄-C₁₂)-diene, for example norbornadiene or 1,5-cyclooctadiene, and

An⁴ is a sulphonate.

Examples of further suitable transition metal compounds include Ni(cod)₂, Pd₂(dibenzylideneacetone)₃, cyclopentadienyl₂Ru, Rh(acetylacetonate)(CO)₂, Ir(pyridine)₂(cod) or multinuclear bridged complexes, for example [Pd(allyl)Cl]₂, [Pd(allyl)Br]₂, [Rh(cod)Cl]₂, [Rh(cod)Br]₂, [Rh(ethene)₂Cl]₂, [Rh(cyclooctene)₂Cl]₂, [Ir(cod)Cl]₂ and [Ir(cod)Br]₂, [Ir(ethene)₂Cl]₂ and [Ir(cyclooctene)₂Cl]₂.

Particularly preferred transition metal compounds are: [Pd(allyl)Cl]₂, [Pd(allyl)Br]₂, [Rh(cod)Cl]₂, [Rh(cod)₂Br [lacuna], [Rh(cod)₂]OTf, [Rh(cod)₂]OMes, [Rh(cod)₂]ONf, RuCl₂(cod), [(cymene)RuCl₂]₂, [(benzene)RuCl₂]₂, [(mesitylene)RuCl₂]₂, [(cymene)RuBr₂]₂, [(cymene)RuI₂]₂, [Ir(cod)₂Cl]₂, [Ir(cod)₂]OTf, [Ir(cod)₂]OMes, [Ir(cod)₂]Onf, [Rh(nbd)₂Br], [Rh(nbd)₂]OTf, [Rh(nbd)₂]OMes, [Rh(nbd)₂]Onf, RuCl₂(nbd), [Ir(nbd)₂]OTf, [Ir(nbd)₂]OMes, [Ir(nbd)₂]ONf, Ir(pyridine)₂(nbd)OTf, [Ru(DMSO)₄Cl₂], [Ru(ACN)₄Cl₂], [Ru(PhCN)₄Cl₂] and [Ru(cod)Cl₂]ₙ.

It should be pointed out that it is necessary when using halide-containing transition metal compounds, for example, to additionally use thallium, silver or potassium sulphonates as defined above in an approximately equimolar amount to the halide present.

To prepare the substances according to the invention, the support material is reacted with compounds of the formula (I).

The weight ratio of compounds of the formula (I) to support material may be, for example and with preference, 0.02:1 to 100:1, particularly preferably 0.1:1 to 5:1 and very particularly preferably 0.1:1 to 1:1.

The reaction temperature may be, for example and with preference, −20 to 100° C., particularly preferably 0 to 80° C. and very particularly preferably 10 to 30° C.

The substances according to the invention may be worked up in a manner known per se by filtration and/or centrifugation and/or sedimentation and optionally subsequent washing with organic solvent, and the washing may be carried out, for example, batchwise or continuously. For storage purposes, the compounds according to the invention are preferably dried.

The substances according to the invention may be used directly as catalyst for asymmetric reactions.

The invention therefore also encompasses catalysts which comprise the substances according to the invention.

The invention also encompasses a process for catalytically preparing enantiomerically enriched compounds, which is characterized in that the catalysts used are those which comprise substances according to the invention.

Preferred processes for preparing enantiomerically enriched compounds are asymmetric hydrogenations, for example hydrogenations of prochiral C═C bonds such as prochiral enamines, olefins, enol ethers; C═O bonds such as prochiral ketones and C═N bonds such as prochiral imines. Particularly preferred asymmetric hydrogenations are hydrogenations of prochiral ketones, in particular α- and β-ketocarboxylic esters.

Preferred α- and β-ketocarboxylic esters are compounds of the formula (IV)

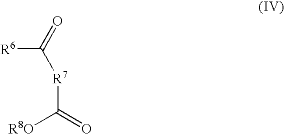

where

R⁶ and R⁸ are each independently C₁-C₁₂-alkyl, C₁-C₁₂-haloalkyl, C₅-C₁₅-arylalkyl or C₄-C₁₄-aryl and

R⁷ is absent or is 1,1-(C₁-C₄-alkylene).

Preferably, R⁶ and R⁸ are each independently optionally chlorinated C₁-C₄-alkyl or phenyl, and

R⁷ is methylene or is absent.

Particularly preferred compounds of the formula (IV) are methyl phenylglyoxylate, methyl benzoylformate and ethyl chloroacetoacetate.

The hydrogenation according to the invention of α- and β-ketocarboxylic esters provides enantiomerically enriched compounds of the formula (V)

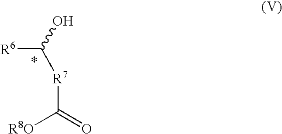

where

* marks a stereogenic centre which is S- or R-configured and

R⁵, R⁶ and R⁷ each have the definitions and areas of preference specified under the formula (V).

In a preferred embodiment of asymmetric hydrogenations according to the invention, the reaction temperature is 0 to 200° C., preferably 10 to 150° C., and the partial hydrogen pressure is, for example, 0.1 to 200 bar, preferably 0.9 to 100 bar and particularly preferably 4 to 30 bar.

Useful solvents for asymmetric hydrogenations according to the invention are in particular aliphatic or aromatic, optionally halogenated hydrocarbons, for example petroleum ether, benzene, toluene, the isomeric xylenes, chlorobenzene, the isomeric dichlorobenzenes, hexane, cyclohexane, dichloromethane or chloroform, ethers such as diethyl ether, diisopropyl ether, dioxane, tetrahydrofuran, methyl tert-butyl ether or ethylene glycol dimethyl ether or ethylene glycol diethyl ether, and also preferably alcohols such as methanol, ethanol and isopropanol.

The weight ratio of catalysts according to the invention to substrate may be, for example, 1:1 to 1:10 000, preferably a ratio of 1:5 to 1:1000.

The advantage of the present invention is that heterogeneous catalysts may be prepared in high yields and in an efficient manner and that these catalysts allow high conversions and enantioselectivities in asymmetric syntheses. It is, therefore, a distinct feature that the compounds of the formula (I), in the case of homogeneous use, surprisingly allow only very low enantioselectivities, if any at all, as the comparative examples hereinbelow show.

The invention is further described with the following illustrative non-limiting examples.

### EXAMPLES

### Example 1

Preparation of [Rh(cod)((S)-2-aminomethyl-1-ethylpyrrolidine]CF₃SO₃

[RhCl(cod]₂ (100 mg, 0.20 mmol) was dissolved in THF (10 ml), AgCF₃SO₃ (104 mg, 0.40 mmol) was added and the solution was stirred for one hour. The solution was subsequently filtered, the filtrate admixed with (S)-2-aminomethyl-1-ethylpyrrolidine (52 mg, 0.40 mmol) and the resulting solution was stirred for one hour. Subsequently, the solution was concentrated under reduced pressure and admixed with hexane (25 ml), and the product precipitated out. The mixture was filtered, and the product was washed with hexane (2×20 ml) and diethyl ether (2×20 ml) and dried under reduced pressure. A yellow powder was obtained (172 mg, 88%).

Anal.: Calculated for C₁₅H₂₈N₂RhBF₄: C, 39.34; H, 5.74; N, 5.74. Found: C, 39.84; H, 5.54; N, 5.69.

¹H NMR (CDCl₃) 1.76-4.3 (28H, amine and olefin).

¹³C NMR (CDCl₃)=12.2 (1), 21.7 (4), 24.4 (5), 45.5 (7), 51.0 (2), 56.5 (3), 67.1 (6), 30.4, 30.7 (CH₂) 79.7, 83.6 (CH).

+ve ESI=339 (M⁺).

### Example 2

Preparation of Heterogenized [Rh(cod)((S)-2-aminomethyl-1-ethylpyrrolidine]CF₃SO₃

The complex from Example 1 was added to dried, calcined MCM-41 (500 mg) and CH₂Cl₂ (20 ml). The mixture was stirred for three hours. Within this time, the colour of the MCM-41 support changed to yellow. Subsequently, the mixture was filtered, the residue was washed with plenty of CH₂Cl₂ until no more complex could be seen to be washed out and the product was dried under reduced pressure.

Anal.: C, 3.77; H, 0.83; N, 0.42.

### Example 3

Preparation of [Rh(cod)((1R,2R)-1,2-diphenylethylenediamine)]CF₃SO₃

[RhCl(cod]₂ (100 mg, 0.20 mmol) was dissolved in THF (10 ml), AgCF₃SO₃ (104 mg, 0.40 mmol) was added and the solution was stirred for one hour. The solution was subsequently filtered, the filtrate admixed with (1R, 2R)-1,2-diphenylethylenediamine (80 mg, 0.4 mmol) and the resulting solution was stirred for one hour. Subsequently, the solution was concentrated under reduced pressure and admixed with hexane (25 ml), and the product precipitated out. The mixture was filtered, and the product was washed with hexane (2×20 ml) and diethyl ether (2×20 ml) and dried under reduced pressure. A yellow powder was obtained (200 mg, 88%).

Anal.: Calculated for C₂₃H₂₈N₂RhF₃SO₃ C, 48.25; H, 4.90; N, 4.90. Found: C, 47.95, H, 4.86; N, 4.60.

¹H NMR (CD₃OD) 1.95 (br m, CH₂ 4H), 2.45 (br m, CH₂, 4H), 4.01 (s, NCH, 2H), 4.23 (m, CH, 2H), 4.35 (m, CH, 2H), 7.1-7.3 (m, Ph, 10H).

¹³C NMR (CD₃OD) 31.5 (CH₂), 66.3 (NCH) 81.4 (CH), 128.5, 129.2, 129.68, 140.5 (Ph).

+ve ESI 423 (M⁺).

### Example 4

Preparation of Heterogenized [Rh(cod)((1R,2R)-1,2-diphenylethylenediamine)]CF₃SO₃

The complex from Example 3 was added to dried, calcined MCM-41 (500 mg) and CH₂Cl₂ (20 ml). The mixture was stirred for three hours. Within this time, the colour of the MCM-41 support changed to yellow. Subsequently, the mixture was filtered, the residue was washed with plenty of CH₂Cl₂ until no more complex could be seen to be washed out and the product was dried under reduced pressure.

Anal.: C, 3.76; H, 0.72; N, 0.39.

### Examples 5 and 6

In a similar manner to Example 3 the following were obtained

5) [Rh(cod)((S)-(2-pyrrolidinemethyl)pyrrolidine)]CF₃SO₃

6) [Pd(allyl)((S)-(2-pyrrolidinemethyl)pyrrolidine)]CF₃SO₃

### Examples 7-16

In a similar manner to Example 4 the following were obtained

7) [Rh(cod)((1R,2R)-1,2-diphenylethylenediamine)]CF₃SO₃ on/in Davison 923

8) [Rh(cod)((1R,2R)-1,2-diphenylethylenediamine)]CF₃SO₃ on/in Davison 634

9) [Rh(cod)((1R,2R)-1,2-diphenylethlenediamine)]CF₃SO₃ on/in Davison 654

10) [Rh(cod)((S)-(2-pyrrolidinemethyl)pyrrolidine)]CF₃SO₃ on/in Davison 923

11) [Rh(cod)((S)-(2-pyrrolidinemethyl)pyrrolidine)]CF₃SO₃ on/in Davison 634

12) [Rh(cod)((S)-(2-pyrrolidinemethyl)pyrrolidine)]CF₃SO₃ on/in Davison 654

In a Similar Manner to Example 2, the Following were Obtained:

13) [Rh(cod)((S)-2-aminomethyl-1-ethylpyrrolidine)]CF₃SO₃ on/in Davison 923

14) [Rh(cod)((S)-2-aminomethyl-1-ethylpyrrolidine)]CF₃SO₃ on/in Davison 634

15) [Rh(cod)((S)-2-aminomethyl-1-ethylpyrrolidine)]CF₃SO₃ on/in Davison 653

16) [Pd(allyl)((S)-(2-pyrrolidinemethyl)pyrrolidine)]CF₃SO₃ on/in MCM 41

### Examples 17 to 44

### Asymmetric Hydrogenations

General Procedure

The asymmetric hydrogenations were carried out in a high-pressure autoclave made of rust-free stainless steel and having a capacity of 150 ml. 10 mg in each case of the homogeneous catalyst or 50 mg in each case of the immobilized catalysts were transferred into the high-pressure autoclave under an inert atmosphere.

The substrate (0.5 g), methanol (30 g) and an internal standard (cyclododecane) were added and the high-pressure autoclave was closed. The high-pressure autoclave and its inlets and outlets were subsequently inertized by flushing with nitrogen three times and, to test the seal, finally placed under a hydrogen pressure of 5 bar. Subsequently, the hydrogen pressure was increased to 20 bar, the high-pressure autoclave was brought to reaction temperature (313 K) and the contents were stirred with a mechanical stirrer at 400 rpm.

An automatic withdrawal valve was used to take samples of the contents, in order to be able to investigate the progress of the reaction. At the end of the reaction, the high-pressure autoclave was cooled for two hours in an ice bath and decompressed, and the products were identified by gas chromatography (GC, Varian, Model 3400 CX) using a chiral column (Chiraldex, 20 m×0.25 mm).

The results of the hydrogenation experiments are compiled in the following tables:

Substrate: methyl benzoylformate

| | Catalyst | | | | | |
|---|---|---|---|---|---|---|
| | from | | t | Conversion | TOF | ee |
| Example | Example | Reaction type | (h) | (%) | (h⁻¹) | (%) |
| 17 | 5 | Homogeneous | 0.5 | 46.2 | 145 | 53 |
| 18 | 10 | Heterogeneous | 0.5 | 92.8 | 643 | 85 |
| 19 | 10 | Heterogeneous | 2.0 | 95.8 | 166 | 94 |
| 20 | 11 | Heterogeneous | 0.5 | 63.0 | 436 | 72 |
| 21 | 11 | Heterogeneous | 2.0 | 91.5 | 159 | 78 |
| 22 | 12 | Heterogeneous | 0.5 | 60.7 | 420 | 65 |
| 23 | 12 | Heterogeneous | 2.0 | 86.9 | 151 | 59 |
| 24 | 1 | Homogeneous | 2.0 | 62 | 46 | 0 |
| 25 | 13 | Heterogeneous | 0.5 | 82.6 | 542 | 82 |
| 26 | 13 | Heterogeneous | 2.0 | 93.3 | 153 | 77 |
| 27 | 14 | Heterogeneous | 0.5 | 67.1 | 440 | 65 |
| 28 | 14 | Heterogeneous | 2.0 | 93.9 | 154 | 61 |
| 29 | 15 | Heterogeneous | 0.5 | 44.6 | 292 | 0 |
| 30 | 15 | Heterogeneous | 2.0 | 86.1 | 141 | 0 |
| 31 | 3 | Homogeneous | 2.0 | 69.9 | 60 | 0 |
| 32 | 7 | Heterogeneous | 0.5 | 77.7 | 596 | 50 |
| 33 | 7 | Heterogeneous | 2.0 | 98.1 | 188 | 79 |
| 34 | 8 | Heterogeneous | 0.5 | 59.7 | 458 | 68 |
| 35 | 8 | Heterogeneous | 1.0 | 75.5 | 290 | 73 |
| 36 | 9 | Heterogeneous | 0.5 | 38.8 | 298 | 0 |
| 37 | 9 | Heterogeneous | 2.0 | 83.1 | 159 | 4 |
| 38 | 6 | Homogeneous | 0.5 | 96.0 | 264 | 55 |
| 39 | 16 | Heterogeneous | 0.5 | 89.8 | 542 | 62 |
| 40 | 16 | Heterogeneous | 2.0 | 98.9 | 149 | 67 |
| 41 | 16 | Heterogeneous | 2.0 | 100 | 151 | 66 |
| | (recycled) | | | | | |

Substrate: methyl phenylglyoxylate

| | Catalyst | | Reaction | | |
|---|---|---|---|---|---|
| | from | Temperature | time | Conversion | ee |
| Example | Example | [° C.] | [h] | [%] | [%] |
| 42 | 3 | 40 | 2 | 82.0 | 0 |
| 43 | 2 | 40 | 2 | 98.9 | 93.3 |
| 44 | 4 | 40 | 2 | 98.3 | 89.1 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Substances comprising at least
one micro-, meso- or macroporous support material and
compounds, adsorbed thereon or therein, of the formula (I) 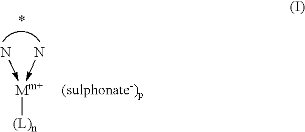
where 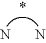
is an enantiomerically enriched chiral nitrogen compound,
(M^{m+}) is a metal having valency m
L is an anionic or uncharged ligand
(sulphonate⁻) is the anion of a sulphonic acid and
p is one or two and
n is one, two, three or four,
with the proviso that m−p−[number of anionic ligands]=0.

2. Substances according to claim 1, characterized in that the support materials have a pore size of 15 to 250 Å.

3. Substances according to claim 1, characterized in that the support materials are silica gels or zeolites of the MOR, X, Y, MCM, ZSM, FAU, MFI, L, BEA, FER, A and SBA, AIPO, MAIPO or SAPO type, and the zeolites are optionally isomorphically substituted.

4. Substances according to claim 1, characterized in that, in formula (I), 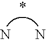
is enantiomerically enriched chiral nitrogen compounds of the formula (II) 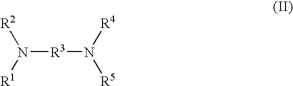
where
R¹, R², R⁴ and R⁵ are each independently hydrogen, C₁-C₈-alkyl, C₅-C₁₅-arylalkyl, C₄-C₁₄-aryl, or NR¹R² and/or NR⁴R⁵ as a whole is a cyclic amino radical having a total of 4 to 20 carbon atoms,
R³ is a divalent radical having 2 to 30 carbon atoms or
R³ and at least one of the radicals R¹, R², R⁴ and R⁵ together are part of a cyclic amino radical having a total of 4 to 20 carbon atoms.

5. Substances according to claim 4, characterized in that
R¹, R², R⁴ and R⁵ are each independently hydrogen, C₁-C₈-alkyl, C₅-C₁₅-arylalkyl or C₄-C₁₄-aryl, or NR¹R² and/or NR⁴R⁵ as a whole is a 5- or 6-membered monocyclic amino radical which is optionally mono-, di-, tri- or tetrasubstituted on the carbon framework by C₁-C₄-alkyl and
R³ is a divalent radical which is selected from the group of C₂-C₈-alkylene which may optionally be further mono- or diubstituted by C₄-C₁₄-aryl radicals, C₅-C₁₅-arylalkylene, C₄-C₁₄-arylene or bis(C₄-C₁₄-arylene) or
R³ and one of the radicals R¹, R², R⁴ and R⁵ together are part of a 5- or 6-membered monocyclic amino radical which is optionally additionally mono-, di-, tri- or tetrasubstituted on the carbon framework by C₁-C₄-alkyl.

6. Substances according to claim 1, characterized in that, in formula (I),
(M^{m+}) is cobalt in the formal oxidation states 0, +2 and +3, rhodium and iridium in the formal oxidation states +1 and +3, nickel, palladium and platinum in the formal oxidation states 0 and +2 or ruthenium in the formal oxidation state +2.

7. Substances according to claim 1, characterized in that, in formula (I),
L is the following types of ligand: monoolefins, diolefins, nitriles, aromatics or anionic ligands.

8. Substances according to claim 1, characterized in that
(sulphonate⁻) is salts of the type R⁶SO₃— where R⁶ is C₁-C₁₂-alkyl, C₁-C₂₀-haloalkyl, C₄-C₁₄-aryl or C₅-C₁₅-arylalkyl.

9. Substances according to claim 1, characterized in that compounds of the formula (I) are those of the formulae (Ia), (Ib), (Ic), (Id), (le) and (If) 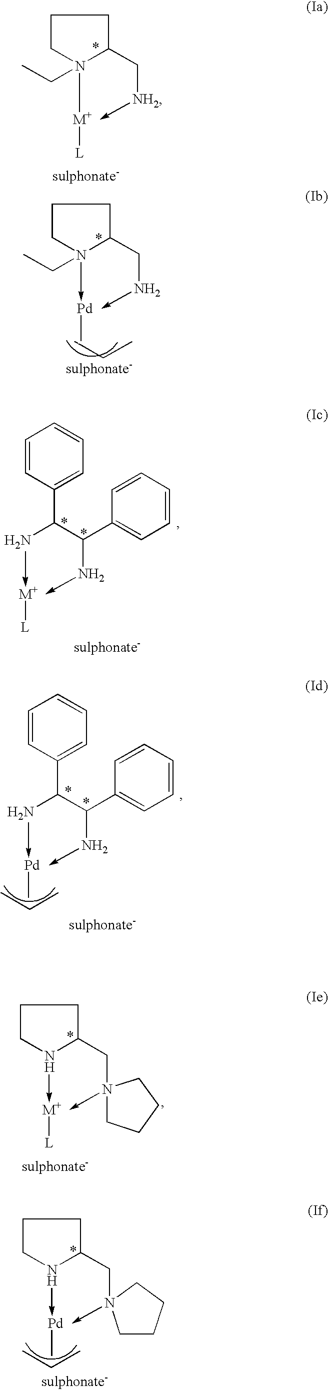
where, in each case,
* marks a stereogenic centre which is either R- or S-configured, with the proviso that mesoforms are excluded (compounds of the formula (Ic) and (Id))
M⁺ is rhodium¹ or iridium¹ and
L is cod or nbd and
sulphonate⁻ is trifluoromethanesulphonate, mesylate or nonafluorobutanesulphonate.

10. Compounds of the formula (I) as defined in claim 1, with the exception of the following compounds:
[Rh(cod)((S)-2-aminomethyl-1-ethylpyrrolidine)] OTf and
[Rh(cod)((1R,2R)-1,2-diphenylethylenediamine)] OTf.

11. A process for conducting asymmetric reactions comprising catalyzing the reactions with substances according to claim 1.

12. Catalysts comprising substances according to claim 1.

13. Process for preparing enantiomerically enriched compounds comprising catalyzing the preparation with catalysts according to claim 12.

14. Process according to claim 13, characterized in that processes for catalytically preparing enantiomerically enriched compounds are asymmetric hydrogenations.

15. Process according to claim 14, characterized in that asymmetric hydrogenations are hydrogenations of prochiral C═C bonds, C═O bonds and C═N bonds.

16. Process according to claim 15, characterized in that hydrogenations of prochiral C═O bonds are hydrogenations of α- and β-keto carboxylic esters.

17. Process according to claim 16, characterized in that α- and β-keto carboxylic esters are those of the formula (IV) 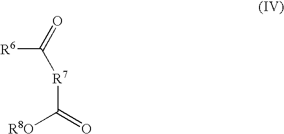
where
R⁶ and R⁸ are each independently C₁-C₁₂-alkyl, C₁-C₁₂-haloalkyl C₅-C₁₅-arylalkyl or C₄-C₁₄-aryl and
R⁷ is absent or is 1,1-(C₁-C₄-alkylene).

18. Process according to claim 14, characterized in that the reaction temperature for asymmetric hydrogenations is 0 to 200° C. and the partial hydrogen pressure is 0.1 to 200 bar.

19. Process according to claim 14, characterized in that the asymmetric hydrogenations is conducted in the presence of solvents which are aliphatic or aromatic, optionally halogenated, hydrocarbons, ethers and/or alcohols.

20. Process according to claim 13, characterized in that the weight ratio of catalysts according to claim 1 to substrate is 1:1 to 1:10 000.
